(19) United States
(12) Patent Application Publication
(10) Pub. No.: US 2010/0312582 A1
(43) Pub. Date: Dec. 9, 2010

(54) SINGLE NUCLEOTIDE POLYMORPHISMS ASSOCIATED WITH DIETARY WEIGHT LOSS

(75) Inventor: Thorkild Ingvor Arrild SØRENSEN, Copenhagen (DK)

Correspondence Address: NIXON & VANDERHYE, PC, 901 NORTH GLEBE ROAD, 11TH FLOOR, ARLINGTON, VA, 22203, US

(73) Assignee: DSM IP ASSETS B.V., Heerlen (NL)

(21) Appl. No.: 12/814,772

(22) Filed: Jun. 14, 2010

Related U.S. Application Data
(63) Continuation-in-part of application No. PCT/EP2008/067978, filed on Dec. 19, 2008.

(30) Foreign Application Priority Data
Dec. 19, 2007 (EP) 07123679.8

(51) Int. Cl. G06Q 50/00; C07H 21/04; C12Q 1/68

(52) U.S. Cl. 705/3; 536/23.1; 435/6

(57) ABSTRACT

The present invention relates to genetic polymorphisms associated with obesity and obesity-related phenotypes and their use in predicting if an individual completes a dietary weight loss intervention program.

### FIELD OF THE INVENTION

The present invention is in the field of obesity. More in particular it relates to genetic polymorphisms and their effect on dietary weight loss intervention programs. Moreover, the present invention pertains to genetic tests and methods using the polymorphisms, particularly methods to predict an obese individual's likelihood to complete a dietary weight loss intervention program.

### BACKGROUND OF THE INVENTION

Obesity is a worldwide epidemic found across all age groups. Especially in industrialized countries, it has increased at a fast rate over the past two decades and is now a worldwide leading public health problem. For example, while in 1996 26% adult Americans were overweight and 10% severely so, currently more than 65% are overweight, with nearly 31% meeting the criteria for obesity. As obesity portends an epidemic of related chronic diseases such as type-2 diabetes, hypertension and cardiovascular events, people with obesity especially people with extreme obesity are at risk for many health problems. The economic cost attributable to obesity in the United States alone has been estimated to be as high as $100 billion per year and includes not only direct health care costs but also the cost of lost productivity in affected individuals.

While diet and lifestyle contribute to obesity and the trend of decreased physical activity and increased caloric intake is probably responsible for the recent rise in obesity, it is important to understand that genetics plays a key role. Each individual's genetic background remains an important determinant of susceptibility to obesity. For instance, half of the population variation in body mass index (BMI), a common measure of obesity, is determined by inherited factors.

Many studies have reported that common genetic variants, usually single nucleotide polymorphisms (SNPs), are associated with an increased risk for obesity. Two approaches have been used to date to find these variants, linkage analysis and association studies. Although on the basis of linkage studies some regions have been repeatedly implicated to play a role in obesity, no genes have been found in these regions that have been seen to contribute to the disease. By using association studies several associations between obesity or obesity-related traits and common genetic variants have been reported. Unfortunately, many of the reported associations have not been consistently replicated.

A gene which has recently been reproducibly associated with obesity is the fat mass and obesity associated (FTO) gene. It was found that some potentially functional SNPs in the FTO locus are strongly associated with early-onset and severe obesity in European populations (see Frayling et al., 2007, Dina et al., 2007, Scuteri et al., 2007) and that certain common genetic variants in the FTO gene are associated with obesity-related traits such as BMI, hip circumference and/or weight. It has been found that the FTO gene encodes a 2-oxoglutarate-dependent nucleic acid demethylase (see Gerken et al., 2007).

Recently, two studies have been published examining the effect of FTO in relation to weight loss. Haupt et al., 2008 examined the effect of variation in rs8050136, a SNP present in the FTO gene (in the same linkage disequilibrium block as SNP rs99390609), on change in anthropometric and metabolic parameters from baseline to the 9-month visit in 204 subjects participating in a 2 year lifestyle intervention program (TULIP). After 9-months, average weight loss was ˜3 kg or ˜1 BMI unit. No effect was found of rs8050136 on weight loss (P=0.38), ΔFM (fat mass; P=0.91) or other anthropometric or metabolic parameters.

Müller et al., 2008 confirmed the association of the risk A allele of rs9939609 with overweight and early onset obesity (one sided p=0.036). However, they did not observe any association of rs9939609 alleles with weight loss or fasting levels of blood glucose, triglycerides and cholesterol.

Altering dietary habits is the cornerstone of weight loss intervention programs for overweight and obese patients. As it is unlikely that one common diet is optimal for all overweight or obese individuals, dietary guidance should be individualized to allow for personalized approaches and recommendations and to increase success rates in these programs. Despite the increasing knowledge of loci and genes associated with obesity and obesity-related traits, no useful genetic variants exist on the basis of which dietary weight loss intervention programs can be tailored for overweight or obese individuals. A recent study even drew the conclusion that common SNPs in a panel of obesity-related candidate genes play a minor role, if any, in modulating weight changes induced by certain diets (see Sørensen et al., 2006).

Given that no predictive information about the genetic response to diet is available, there is a need in the art for identifying genetic variants that predict the response of an overweight or obese individual to a dietary weight loss intervention program, for instance SNPs that predict the likelihood that an overweight or obese individual completes such a program and thus benefits from the program. The present invention meets these needs.

### SUMMARY OF THE INVENTION

It was found in accordance with the present invention that markers exist that are associated with the likelihood that an individual, such as an overweight or obese individual, completes a certain dietary weight loss intervention program. The term “associated with” in connection with the relationship between a genetic characteristic, e.g., a marker, allelic variant, haplotype, or polymorphism, and a trait means that there is a statistically significant level of relatedness between them based on any generally accepted statistical measure of relatedness. Those skilled in the art are familiar with selecting an appropriate statistical measure for a particular experimental situation or data set. Accordingly, the present invention is directed to methods wherein use is made of the genetic characteristics. The invention also provides kits to determine whether an individual is likely to complete a specific diet on the basis of analysis of genetic markers e.g. SNPs. The markers can for instance be found in genes associated with overweight, obesity or obesity-related traits. The resulting information can be used to classify individuals such as overweight or obese individuals based on their genetic tendency to complete or fail to complete certain types of diet. This will help professionals in the field of weight management to improve targeting these individuals with appropriate (nutritional) advice regarding their weight management. As a result thereof, the success rate of dietary weight loss intervention programs will increase.

### DETAILED DESCRIPTION OF THE INVENTION

The invention is based on the finding that obese individuals carrying at least one A allele of the SNP rs9939609 have a higher tendency to complete low fat/high carbohydrate diets than high fat/low carbohydrate diets, while obese individuals being homozygous for the common T allele of the SNP rs9939609 have a higher tendency to complete high fat/low carbohydrate diets than low fat/high carbohydrate diets. The nucleotide sequence of the SNP rs9939609 is shown in SEQ ID NO. 1 (AGGTTCCTTGCGACTGCTGTGAAT TT[A/T]GTGATGCACTTGGATAGTCTCTGTT). The polymorphism may thus be useful in predicting the outcome of weight loss intervention programs, particularly programs having a component of dietary intervention e.g. diets. The polymorphism may be part of a haplotype which may have an association link with the likelihood of an individual to complete or fail to complete a certain dietary weight loss intervention program. As used herein, “haplotype” refers to a set of alleles found at linked polymorphic sites on a single chromosome. The linked sites may include part of a gene, an entire gene, several genes, or a region devoid of genes (but which perhaps contains a DNA sequence that regulates the function of nearby genes). The haplotype preserves information about the phase of the polymorphic nucleotides, that is, which set of variances were inherited from one parent (and are therefore on one chromosome) and which from the other. In a preferred embodiment the programs comprise dietary intervention either alone or as a major component. Next to suitable diets, i.e. personalized diets based on the genetic profile of an individual, weight loss intervention programs may however also include other components such as e.g. drug treatment, surgical treatment e.g. liposuction, behavioural therapy, increase in physical activity and dietary supplement treatment.

In view of the fact that obese individuals that carry at least one A allele of the SNP rs9939609 have more difficulty in completing a high fat/low carbohydrate diet than a low fat/high carbohydrate diet, the identification of an obese individual carrying an A allele can help weight management professionals to design suitable dietary weight loss intervention programs for these individuals. Mutatis mutandis, this also goes for obese individuals that are homozygous for the common T allele of the SNP rs9939609.

In an aspect the invention relates to the use of at least one genetic marker such as a polymorphism, e.g. a SNP, for determining the likelihood that an individual completes or fails to complete a dietary weight loss intervention program. In other words, the data provided herein show that a correlation, association, linkage or other relation between a specific marker and a risk of failing to complete a specific type of diet can be established. The marker determines the likelihood that an individual completes or fails to complete a dietary component of the intervention program such as a diet. Diets used in dietary weight loss intervention programs designed to treat individuals are well known to the skilled person. Preferred diets in the light of the present invention include but are not limited to high fat/low carbohydrate diets or low fat/high carbohydrate diets. The high fat/low carbohydrate or low fat/high carbohydrate diets may be hypo-energetic diets (also called hypo-caloric diets). In an embodiment the individual is overweight or obese. An “individual” as used in the present application refers to a vertebrate, preferably a mammal, more preferably an animal such as a domestic animal (e.g. a dog or cat), and most preferably a human.

An “overweight individual”, as used herein, refers to an individual fulfilling the normal definition of overweight individual as defined by the medical knowledge at the time of diagnosis. Useful criteria for defining an individual as overweight include, but are not limited to, a body mass index (BMI) of 25-29.9, male individual with a waist measurement greater than 40 inches (102 cm), female individual with a waist measurement greater than 35 inches (88 cm), and all individuals with a waist-to-hip ratio of 1.0 or higher. An “obese individual”, as used herein, refers to an individual fulfilling the normal definition of obese individuals as defined by the medical knowledge at the time of diagnosis. Useful criteria for defining an individual as obese include, but are not limited to, a body mass index (BMI) of 30 or higher.

A “hypo-energetic (hypo-caloric) diet” as used herein means a diet wherein the daily energy intake is less than the daily energy requirement, e.g. a diet with an energy deficiency of at least 100, 200, 300, 400, 600, 800, 1000, 1200, 1500 or 2000 kcal/day. “High fat” diets as used herein means diets having at least 30%, preferably at least 40%, more preferably 40-45% of energy from fat. “Low fat” diets as used herein means diets having less than 30%, preferably less than 25%, more preferably 20-25% of energy from fat. “Low carbohydrate” diets as used herein means diets having less than 50%, preferably less than 45%, more preferably 40-45% of energy from carbohydrate. “High carbohydrate” diets as used herein means diets having at least 50%, preferably at least 60%, more preferably 60-65% of energy from carbohydrate. The diets may further contain other components such as e.g. proteins. The diets may have e.g. 15% of energy from proteins. Preferably, the individuals on the dietary intervention program do not consume alcohol. Where exclusion of alcohol is not possible, intake should be minimal, with an upper limit of two glasses (2×150 ml) in total. Energy from alcohol should be subtracted from total energy intake and thereafter macronutrient intake should be calculated on the remaining energy. Where possible, viscous soluble fibres should be avoided in the diets, since they are thought to have the greatest impact on glucose and lipid metabolism (e.g. oats and guar gum). Furthermore, it may be attempted to standardise other sources of soluble fibre within the diets (e.g. fruit and vegetables, especially legumes). Individuals participating in dietary intervention programs may be encouraged to consume equal amounts of polyunsaturated, monounsaturated and saturated fats by ensuring incorporation of olive oil (or equivalent) and sunflower oil (or equivalent) into each day's choices (in addition to saturated fat predominately from meat and dairy products). They may avoid using food products including specialist margarines which contain added plant sterols, omega-3 fatty acids or soy compounds, and soy based products. Furthermore, they may be encouraged to consume oily fish at least once a week within the fat restriction of the diet and they may attempt to maintain comparable ratios of simple sugars to complex carbohydrates. Individuals who are already taking vitamin and mineral supplementation before starting the dietary intervention program may continue taking the same dose throughout the program and this intake may be included in the intake analysis.

In an embodiment of the invention the marker is present in a locus, gene or gene cluster associated with an obesity-related phenotype. As used herein, “phenotype” refers to any observable or otherwise measurable physiological, morphological, biological, biochemical or clinical characteristic of an individual. Of course, a combination of markers can be used in the methods, kits, uses, etc of the present invention. The markers may be present in coding (exons) but may also be present in non-coding regions (intron and intergenic regions). They may be present in different genes e.g. one marker in the FTO gene (e.g. SNP rs9939609) and another marker in e.g. the MC4R gene. If more than one marker is used, the markers may be in linkage disequilibrium with one another, preferably in non-tight linkage disequilibrium. “Linkage disequilibrium” or “allelic association” means the preferential association of a particular allele or genetic marker with a specific allele or genetic marker at a nearby chromosomal location more frequently than expected by chance for any particular allele frequency in the population. Linkage disequilibrium may result from natural selection of certain combination of alleles or because an allele has been introduced into a population too recently to have reached equilibrium (random association) between linked alleles. A marker in linkage disequilibrium with disease predisposing variants can be particularly useful in detecting susceptibility to disease (or association with sub-clinical phenotypes), notwithstanding that the marker does not cause the disease. Methods to determine linkage disequilibrium are well known to the skilled artisan. The present invention thus also pertains to methods and uses comprising determining in vitro the genotype of the SNP rs9939609, and/or at least one other SNP in DNA taken from an individual. This other SNP may be in linkage disequilibrium with SNP rs9939609.

Obesity-related phenotypes include but are not limited to body weight, BMI, percent fat mass (FM), percent fat-free mass (FFM), waist circumference (WC), insulin secretion (HOMA-β), insulin resistance (HOMA-IR), fasting energy expenditure (EE), serum triglycerides, cholesterol, and glucose, to name just a few. Genes associated with these phenotypes have been found (see Obesity: Genomics and postgenomics, Eds: Clement and Sørensen, Informa Healthcase, first edition, 2007). In a preferred embodiment the marker, e.g. SNP, is present in the FTO gene, preferably in intron 1 of the FTO gene. In a preferred embodiment the marker is the SNP rs9939609. SNP rs9939609, located in the FTO gene, has been found strongly and reproducibly associated with risk of being overweight and obese in white Europeans. The A allele was associated with higher BMI (increase of ˜0.4 kg/m²; P=2×10⁻²⁰ for each additional copy of the A allele), body weight (˜1.2 kg; P=4×10⁻¹⁷), waist circumference (˜1 cm; P=4×10⁻⁹), and subcutaneous fat mass assessed by skin fold in adults and fat mass assessed by DEXA in children. More information about the FTO gene, protein and SNPs can be found in Frayling et al., 2007, Dina et al., 2007, Scuteri et al., 2007 and Gerken et al., 2007. It is to be understood that any marker that is in linkage disequilibrium with rs9939609 can also be used in the various aspects and embodiment of the present invention. These markers do not necessarily have to be present in the same locus, gene or gene cluster. They may be part of other more distant genes. However, they should be in linkage. “Linkage” describes the tendency of genes, alleles, loci or genetic markers to be inherited together as a result of their location on the same chromosome, and can be measured by percent recombination between the two genes, alleles, loci or genetic markers that are physically-linked on the same chromosome. Loci occurring within 50 centimorgan of each other are linked. Some linked markers occur within the same gene or gene cluster.

In a further aspect, the invention pertains to the use of at least one marker for determining a diet an individual is most likely to complete. In other words, the marker may be used for selecting an optimal diet for an individual. “Optimal” means, among others, that the individual should remain on the diet and complete it. This way he will benefit from the diet. On the basis of a correlation, association, linkage or other relation between a genetic marker and the likelihood to complete and/or remain on a specific diet, a suitable diet can be communicated, prescribed, suggested and/or recommended to an individual and/or added to an individual's food or diet. Preferably, the marker is a genetic marker such as a polymorphism, e.g. a SNP.

As used herein “polymorphism” refers to DNA sequence variation in the cellular genome of an individual, typically with a population frequency of more than 1%. A polymorphic marker or site is the locus at which genetic variation occurs. Preferred markers have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats, hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wild-type form. Diploid organisms may be homozygous or heterozygous for allelic forms. A SNP occurs at a polymorphic site occupied by a single nucleotide. A SNP usually arises due to substitution of one nucleotide for another at the polymorphic site, but it can also arise from an insertion or deletion of a nucleotide relative to a reference allele.

In an embodiment the invention is concerned with a method for assessing whether an individual will benefit from a low fat/high carbohydrate diet or a high fat/low carbohydrate diet, the method comprising the step of determining the genotype of the SNP rs9939609 in the FTO gene, wherein (i) the presence of either one or two A allelic forms of the SNP rs9939609 is indicative of a increased likelihood that the individual completes a low fat/high carbohydrate diet compared to a high fat/low carbohydrate diet and, (ii) the absence of an A allelic form of the SNP rs9939609 is indicative of a increased likelihood that the individual completes a high fat/low carbohydrate diet compared to a low fat/high carbohydrate diet. The individual benefits from the specific diet as he will more likely remain on it and complete it. In an embodiment the individual is overweight or obese. In a further embodiment the individual is a white European. In a further embodiment the high fat/low carbohydrate or low fat/high carbohydrate diet is a hypo-energetic diet.

In an embodiment the present invention also provides a method for predicting the likelihood that an individual completes a dietary weight loss intervention program, the method comprising the steps of a) obtaining a biological sample comprising nucleic acid of the individual, and b) genotyping the nucleic acid for the single nucleotide polymorphism (SNP) rs9939609 in the FTO gene, wherein (i) the presence of either one or two A allelic forms of the SNP rs9939609 is indicative of a decreased likelihood that the individual completes a dietary weight loss intervention program wherein the individual is treated with a high fat/low carbohydrate diet compared to a dietary weight loss intervention program wherein the individual is treated with a low fat/high carbohydrate diet, and (ii) the absence of an A allelic form of the SNP rs9939609 is indicative of a decreased likelihood that the individual completes a dietary weight loss intervention program wherein the individual is treated with a low fat/high carbohydrate diet compared to a dietary weight loss intervention program wherein the individual is treated with a high fat/low carbohydrate diet. In the methods and uses of the present invention the occurrence of the A allelic form of the SNP rs9939609 may be assessed by contacting a nucleic acid derived from the genome of an individual with a first oligonucleotide that anneals with higher stringency with the A allelic form of the polymorphism than with the T allelic form of the polymorphism and assessing annealing of the first oligonucleotide and the nucleic acid, whereby annealing of the first oligonucleotide and the nucleic acid is an indication that the genome of the individual comprises the A allelic form of the polymorphism. The method may be extended by assessing the occurrence of the T allelic form of the polymorphism by contacting the nucleic acid with a second oligonucleotide that anneals with higher stringency with the T allelic form of the polymorphism than with the A allelic form of the polymorphism and assessing annealing of the second oligonucleotide and the nucleic acid, whereby annealing of the second oligonucleotide and the nucleic acid is an indication that at least one allele of the FTO gene in the genome of the individual does not comprise the A allelic form of the polymorphism. The first and second oligonucleotides may be attached to a support. It may be the same support.

“Biological sample” as used in the present invention encompasses a variety of sample types which can be used as source material for isolating nucleic acids. They include, but are not limited to, solid materials (e.g., tissue, tissue cultures or cells derived there from and the progeny thereof, hair follicle samples, biopsy specimens, buccal cells provided by a swab, skin and nose samples) and biological fluids (e.g. urine, fecal material, blood, semen, amniotic fluid, tears, saliva, sputum, sweat, mouth wash). Any biological sample from a human individual comprising even one cell comprising nucleic acid can be used in the methods of the present invention. The term also includes samples that have been manipulated in any way after their procurement, such as by treatment with reagents, solubilisation, or enrichment for certain components, such as proteins or polynucleotides. The methods and uses of the present invention are preferably conducted on a sample that has previously been removed from the individual and do preferably not involve diagnosis practiced on the human body.

Nucleic acid molecules as used herein refers to polymeric forms of nucleotides and includes both sense and antisense strands of RNA, cDNA, genomic DNA, and synthetic forms and mixed polymers of the above, with genomic DNA being preferred. A nucleotide refers to a ribonucleotide, deoxynucleotide or a modified form of either type of nucleotide. The term also includes single- and double-stranded forms of DNA. In addition, a polynucleotide may include either or both naturally-occurring and modified nucleotides linked together by naturally-occurring and/or non-naturally occurring nucleotide linkages. The nucleic acid molecules may be modified chemically or biochemically or may contain non-natural or derivatized nucleotide bases, as will be readily appreciated by those of skill in the art. Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. A reference to a nucleic acid sequence encompasses its complement unless otherwise specified. Thus, a reference to a nucleic acid molecule having a particular sequence should be understood to encompass its complementary strand, with its complementary sequence. The complementary strand is also useful, e.g., for antisense therapy, hybridization probes and PCR primers.

Nucleic acids can be isolated from a particular biological sample using any of a number of procedures, which are well-known in the art, the particular isolation procedure chosen being appropriate for the particular biological sample. Methods of isolating and analyzing nucleic acid variants as described above are well known to one skilled in the art and can be found, for example in the Molecular Cloning: A Laboratory Manual, 3rd Ed., Sambrook and Russel, Cold Spring Harbor Laboratory Press, 2001 and Current Protocols in Molecular Biology Volumes I-III, 4^{th} edition, Ausubel et al., John Wiley and Sons, 1995. Many of the methods require amplification of nucleic acid from target samples. This can be accomplished by techniques such as e.g. PCR, ligase chain reaction, nucleic acid based sequence amplification, self-sustained sequence replication and transcription amplification. Genetic markers such as the SNPs can be detected from the isolated nucleic acids using techniques including DNA hybridization methods (e.g. Southern Blotting, FISH), direct sequencing with radioactively, enzymatically, luminescently or fluorescently labelled primers (manually or automated), restriction fragment length polymorphism (RFLP) analysis, heteroduplex analysis, single strand conformational polymorphism (SSCP) analysis, denaturing gradient gel electrophoresis (DGGE), temperature gradient gel electrophoresis (TGGE), use of linked genetic markers, mass spectrometry e.g. MALDI-TOF, and chemical cleavage analysis to name just a few. Of course DNA MicroArray technology suitable for detecting genetic markers such as SNPs can also be used. All methods are explained in detail, for example, in the Molecular Cloning: A Laboratory Manual, 3rd Ed., Sambrook and Russel, Cold Spring Harbor Laboratory Press, 2001.

Primers used may be oligonucleotides hybridizing specifically with one allele. They are called allele-specific oligonucleotides. In the allele-specific PCR methodology, a target DNA is preferentially amplified only if it is completely complementary to the 3′-terminus of a specific PCR amplification primer. The 3′-terminus of the primer is designed so as to terminate at, or within one or two nucleotides of a known mutation site within the target DNA to which it possesses a complementary sequence. Under the appropriate reaction conditions, the target DNA is not amplified if there is a single nucleotide mismatch (e.g., a nucleotide substitution caused by a mutation) or a small deletion or insertion, at the 3′-terminus of the primer. Accordingly, allele-specific PCR may be utilized to detect either the presence or absence of (at least) a single nucleotide mismatch between the primer sequence (which is complementary to a pre-selected target sequence) and a nucleic acid within the sample. Amplification of the target sequence is indicative of a lack of even a single mismatched nucleotide. The markers in the present invention are preferably analyzed using methods amenable for automation. Primer extension analysis can be performed using any method known to one skilled in the art. Oligonucleotides, probes and/or primers may be naturally occurring or synthetic, but are typically prepared by synthetic means. They may be immobilized on a solid support. For instance, oligonucleotides, probes and/or primers as described herein can be used as a DNA chip. The chip may contain a primer corresponding to a single allelic form of a marker but may also contain a primer corresponding to both allelic forms of a marker. It may even comprise primers for different markers. The appropriate length of an oligonucleotide, probe and/or primer depends on its intended use but typically ranges from 10 to 75, preferably 15 to 40 nucleotides. Short primer molecules generally require cooler temperatures to form sufficiently stable hybrid complexes with the template. A primer need not reflect the exact sequence of the template but must be sufficiently complementary to hybridize with a template. Conditions suitable for hybridization are generally known in the art and will be apparent to the skilled artisan. A non-limiting example of stringent hybridization conditions is hybridization in 6× sodium chloride/sodium citrate (SSC) at about 45° C., followed by one or more washes in 0.2×SSC, 0.1% SDS at 50-65° C. Stringent conditions can for instance be found in Molecular Cloning: A Laboratory Manual, 3rd Ed., Sambrook and Russel, Cold Spring Harbor Laboratory Press, 2001 and Current Protocols in Molecular Biology, John Wiley & Sons, N.Y. (1989), 6.3.1-6.3.6. The term “primer site” refers to the area of the target DNA to which a primer hybridizes. The term “primer pair” means a set of primers including a 5′-upstream primer that hybridizes with the 5′-end of the DNA sequence to be amplified and a 3′-downstream primer that hybridizes with the complement of the 3′-end of the sequence to be amplified.

As used herein, “genotype” refers to the genetic constitution of an individual. More specifically, “genotyping” as used herein refers to the analysis of DNA in a sample obtained from a subject to determine the DNA sequence in a specific region of the genome, e.g. a locus that influences a trait. It may refer to the determination of DNA sequence at one or more polymorphic sites and/or determination of allelic patterns of an individual. The genotyping may be performed using a micro-array or a multi-well plate in for instance a laboratory or hospital. It may thus involve the use of a gene/DNA chip or a strip or solid surface comprising one or more nucleic acid molecules.

A further aspect of the invention pertains to a method for diagnosing an individual as being likely to complete a dietary weight loss intervention program, the method comprising the steps of a) determining the genotype of the SNP rs9939609 in the FTO gene, and b) diagnosing the individual as being likely to complete a dietary weight loss intervention program wherein the individual is treated with a low fat/high carbohydrate diet, if the genotype of the SNP rs9939609 in the FTO gene is NT or NA, and diagnosing the individual as being likely to complete a dietary weight loss intervention program wherein the individual is treated with a high fat/low carbohydrate diet, if the genotype of the SNP rs9939609 in the FTO gene is T/T.

A method of assessing the desirability of treating an individual with a low fat/high carbohydrate or a high fat/low carbohydrate diet, the method comprising assessing occurrence of the SNP rs9939609 in the FTO gene of the individual, wherein occurrence of an A allelic form of the SNP is an indication that it is more desirable to treat the individual with a low fat/high carbohydrate diet than with a high fat/low carbohydrate diet and wherein absence of an A allelic form of the SNP (presence of the T/T alleles) is an indication that it is more desirable to treat the individual with a high fat/low carbohydrate diet than with a low fat/high carbohydrate diet, is a further aspect of the present invention. In other words, the present invention provides a method of assessing the advisability that a individual should employ a dietary weight loss intervention program comprising either a high fat/low carbohydrate diet or a low fat/high carbohydrate diet, the method comprising assessing the occurrence in the genome of the individual of the A allelic form of SNP rs9939609, whereby presence of this form of the polymorphism is an indication that the individual should employ low fat/high carbohydrate diet while absence of the A allelic form of the polymorphism is an indication that the individual should employ high fat/low carbohydrate diet.

The invention further provides a method of assessing the desirability of supplementing the food of an individual with either a high fat/low carbohydrate diet or a low fat/high carbohydrate diet, the method comprising assessing occurrence in the genome of the individual of the A allelic form of the SNP rs9939609, whereby occurrence of a copy of the polymorphism is an indication that it is more desirable to supplement the individual's food with a low fat/high carbohydrate diet than that of an individual whose genome does not comprise the A allelic form of the polymorphism. In an individual wherein the A allelic form is absent, it is more desirable to supplement the individual's food with a high fat/low carbohydrate diet than that of an individual whose genome does comprise an A allelic form of the polymorphism.

The invention is also directed to a method of determining whether an individual is a suitable candidate for a low fat/high carbohydrate or a high fat/low carbohydrate dietary weight loss intervention program, the method comprising the steps of genotyping the SNP rs9939609 in the FTO gene in a nucleic acid sample of the individual, wherein the occurrence of an A allelic form of the SNP is indicative of the individual being a suitable candidate for a low fat/high carbohydrate dietary weight loss intervention program and wherein the absence of an A allelic form of the SNP is indicative of the individual being a suitable candidate for a high fat/low carbohydrate dietary weight loss intervention program.

In yet a further aspect the invention provides a method of determining whether an individual has an increased predisposition to complete a high fat/low carbohydrate diet or a low fat/high carbohydrate diet, the method comprising the step of a) isolating from the individual a nucleic acid comprising the single nucleotide polymorphism (SNP) rs9939609, and b) determining the allelic form of the SNP, wherein the presence of at least one A allelic form of the SNP indicates that the individual has an increased likelihood to complete a low fat/high carbohydrate diet compared to a high fat/low carbohydrate diet and wherein the absence of an A allelic form of the SNP indicates that the individual has an increased likelihood to complete a high fat/low carbohydrate diet compared to a low fat/high carbohydrate diet.

In another aspect the invention relates to a method of assessing the advisability that an individual should employ a high fat/low carbohydrate diet or a low fat/high carbohydrate diet, the method comprising the step of assessing occurrence in the individual's genome of the SNP rs9939609, whereby occurrence of at least one A allelic form of the SNP is an indication that the individual should employ a low fat/high carbohydrate diet and absence of an A allelic form of the SNP is an indication that the individual should employ a high fat/low carbohydrate diet.

In the methods of the invention the high fat/low carbohydrate or low fat/high carbohydrate diet may be a hypo-energetic diet.

The invention also relates to a method of correlating a specific allelic form of a SNP in a gene of an individual with the increased likelihood of the individual to complete a dietary weight loss intervention program, comprising a) identifying an individual (individual A) having completed a dietary weight loss intervention program, b) determining the allelic form of the SNP in the gene of individual A, c) comparing the allelic form of the SNP in the gene of individual A with the allelic form of the SNP in the gene of an individual having failed to complete the dietary weight loss intervention program (individual B), and d) if the allelic form of the SNP differs between individual A and individual B, correlating the specific allelic form of the SNP in the gene of individual A with an increased likelihood of an individual to complete a dietary weight loss intervention program.

In the methods of the invention the individual can be an overweight or obese individual. In a further embodiment the individual is a white European.

The steps of obtaining a sample from an individual, determining the genotype of a SNP, e.g. SNP rs9939609 in the FTO gene, and linking the genotype of the SNP to a specific type of diet or dietary weight intervention program that is more or less suitable for the individual can be done by one party, however, the steps can also be performed by two or even more distinct parties.

Another aspect of the invention is directed to a kit for use in a method or use of the present invention. The kit comprises at least one primer or primer pair suitable for determining (or being associated with) the likelihood that an individual such as an overweight or obese individual completes or fails to complete a dietary weight loss intervention program, more in particular a dietary component thereof such as a diet. In an embodiment the invention is directed to a kit for use in a method or use according to the invention, the kit comprising at least one primer or primer pair for genotyping a marker in a gene or locus associated with obesity or an obesity-associated phenotype such as the FTO gene. Preferably, the marker is SNP rs9939609 in the FTO gene. The primers may be suitable for nucleic acid sequence amplification. Often the kits contain one or more primers or primer pairs hybridizing to different forms of a polymorphism, e.g. a primer or primer pair capable of hybridizing to the A allelic form of SNP rs9939609 and a primer or primer pair capable of hybridizing to the T allelic form of SNP rs9939609. Moreover, kits according to the invention may comprise instructions explaining correlation of the genotype to either increased or decreased likelihood of completion of a specific type of diet such as a high fat/low carbohydrate diet or a low fat/high carbohydrate diet. In case the marker is SNP rs9939609, the instructions may explain that detection of at least one A allelic form of the SNP is indicative of the individual as being more likely to respond to (more susceptible for) a low fat/high carbohydrate dietary weight loss intervention program than to a high fat/low carbohydrate dietary weight loss intervention program and that detection of no A allelic form of the SNP (e.g. T/T allelic forms) is indicative of that individual being more likely to respond to (more susceptible for) a high fat/low carbohydrate dietary weight loss intervention program than to a low fat/high carbohydrate dietary weight loss intervention program. Optional additional components of the kit include, for example, restriction enzymes, reverse transcriptase or polymerase, a positive control, a negative control, at least a further primer pair suitable for detecting (other) markers, appropriate buffers for reverse transcription, PCR and/or hybridization reactions, means used to label and nucleotide mix for the PCR reaction. The kits of the invention may thus also comprise one or more primers, primer pairs, probes and/or oligonucleotides suitable for detecting markers such as SNPs which is/are in linkage disequilibrium with SNP rs9939609. In addition, a kit according to the present invention may contain instructions for carrying out the methods as well as a listing of the obesity-associated alleles and haplotypes relevant in view of the present invention. The components of the kit may be either in dry form in a tube or a vial or dissolved in an appropriate buffer.

The present invention employs, unless otherwise indicated, conventional (recombinant) techniques of molecular biology, immunology, microbiology, biochemistry and cell biology which are well within the skill of a person skilled in the art. All publications and references cited in the present application are incorporated by reference in their entirety for any purpose.

Furthermore, in an embodiment the present invention relates to the use of a low fat/high carbohydrate diet in an individual which has been identified as having at least one A allelic form of the SNP rs9939609 in the FTO gene. Moreover, in an embodiment the present invention relates to the use of a high fat/low carbohydrate diet in an individual which has been identified as having no A allelic form of the SNP rs9939609 in the FTO gene. The specific diet can be used in a dietary weight intervention program for the individual. In the present invention is also encompassed the use of a low fat/high carbohydrate diet in the manufacture of a medicament for the treatment and/or prevention of obesity in an individual which has been identified as having at least one A allelic form of the SNP rs9939609 in the FTO gene. It also relates to the use of a high fat/low carbohydrate diet in the manufacture of a medicament for the treatment and/or prevention of obesity in an individual which has been identified as having no A allelic form of the SNP rs9939609 in the FTO gene. In a further aspect the invention relates to the use of a low fat/high carbohydrate diet or high fat/low carbohydrate diet in the making of a dietary weight intervention program suitable for treating an individual which has been identified as having at least one A allelic form of the SNP rs9939609 in the FTO gene or no A allelic form of the SNP rs9939609 in the FTO gene, respectively. In an embodiment the individual is obese.

In an embodiment the present invention also relates to computer systems and computer readable media for storing data according to the present invention. Computer readable media mean media that can be read and accessed directly by a computer including but not being limited to magnetic storage media e.g. floppy discs, hard disc storage media and magnetic tapes; optical storage media e.g. CD-ROM; electrical storage media e.g. RAM and ROM; and hybrids of these categories e.g. magnetic/optical storage media. The data can be stored in one or more databases and include information relating to markers e.g. SNPs such as SNP rs9939609 suitable for determining the likelihood that an individual completes or fails to complete a dietary weight loss intervention program. The databases may further include information regarding the nature of the marker (e.g. the base occupying a polymorphic position in a reference allele as well as in a non-reference allele), the location of the marker (e.g. by reference to for example a chromosome or distance to known markers within the chromosome), the level of association of the marker with obesity, the frequency of the marker in the population or a subpopulation, the association of the marker with other markers as well as all relevant information about the other markers. It may also include sequences of 10-100 contiguous bases, or their complements, comprising a polymorphic position. The databases may also contain personal information of individuals originating from interviews, questionnaires or surveys as well as relevant medical information originating from doctors, physicians, dieticians, nutritionists or genetic counsellors. In addition, the databases may comprise information regarding all types of diets, dietary components and dietary weight loss intervention programs (including composition, price, dosage, etc). It may even comprise information regarding which diet, dietary component and dietary weight loss intervention program is suitable and/or not suitable for an individual on the basis of its genetic profile. The databases may comprise information from one individual but also from a group of individuals (e.g. a specific population or subpopulation). The databases may be used in the methods and uses of the present invention. Typically, genetic data from an individual will be introduced into the computer system by means of electronic means, for example by using a computer. Next, the genetic data are compared to the data in the databases comprising information relating to genetic markers. On the basis of the comparison the likelihood of an individual to complete a dietary weight loss intervention program can be determined and, optionally, a suitable personalized diet can be advised. The invention also provides a computer program comprising program code means for performing all the above steps when said program is run on a computer. Also provided is a computer program product comprising program code means stored on a computer readable medium for performing the methods and uses of the invention when said program is run on a computer. A computer program product comprising program code means on a carrier wave that, when executed on a computer system, instruct the computer system to perform the above steps is additionally provided. The invention also provides an apparatus arranged to perform the above steps. The apparatus typically comprises a computer system, such as a PC. In one embodiment, the computer system comprises means for receiving genetic data from an individual, a module for comparing the data with a database comprising information relating to genetic markers, and means for determining on the basis of said comparison the likelihood that an individual will complete or fail to complete a dietary weight loss intervention program and optionally even means to determine a suitable diet, dietary component or dietary weight loss intervention program for an individual. Access to the databases can be accomplished electronically, e.g. via a computer (PC or laptop), mobile phone, personal digital assistance, internet, handheld but the information in the databases can also be provided in paper form. People having access to the databases may be the individuals themselves, physicians, nutritionists, doctors, dieticians, and even restaurants and supermarkets. Access may be complete or limited to certain data only.

### EXAMPLES

To illustrate the invention, the following examples are provided. These examples are not intended to limit the scope of the invention.

### Example 1

### Aim

A 10-week dietary weight loss intervention study was done to examine the joint effects of the genotype of SNP rs9939609 present in the FTO gene and fat and carbohydrate (CHO) content of hypo-energetic diets on changes in obesity-related phenotypes. The intervention provided data useful in understanding the mechanisms through which rs9939609 affects changes in obesity-related phenotypes and data regarding the joint effect of SNP rs9939609 and macronutrient composition of hypo-energetic diets on compliance with the 10-week dietary weight loss intervention program as measured by drop-out from the weight loss intervention program.

### Cohort Description

In a 10-week, European, multi-centre dietary intervention study 771 weight stable, obese (BMI >=30 kg/m²), but otherwise healthy men and women were randomised to a low fat/high carbohydrate (20-25% of total energy from fat; 60-65% of total energy from carbohydrate; 15% of total energy from protein) or high fat/low carbohydrate (40-45% of total energy from fat; 40-45% of total energy from carbohydrate; 15% of total energy from protein), hypo-energetic diet. Both diets were designed to provide 600 kcal/d (1 kilocalorie (kcal)=4.2 kilo joule (kJ)) less than the individually estimated energy requirement based on an initial resting metabolic rate multiplied by 1.3. Subjects were given oral and written instructions relating to these targets based on either a template or exchange system. Instructions were also given to minimize differences between the two diets in other components such as sources and type of fat, amount and type of fiber, type of carbohydrate, fruit and vegetables, and meal frequency and participants were requested to abstain from alcohol consumption. Dietary instructions were reinforced weekly.

### Selection of Patients

Obese subjects were recruited from May 2001 until September 2002. Inclusion criteria were: body mass index (kg/m²) 30 and age 20-50 years. Exclusion criteria were: weight change >3 kg within the 3 months prior to the study start, hypertension, diabetes or hyperlipidemia treated by drugs, untreated thyroid disease, surgically or drug-treated obesity, pregnancy, and participation in other trials, and alcohol or drug abuse.

771 obese white Europeans (579 women) were included and randomized to one of the two intervention diets by stratified block randomization. The randomization list was computer generated at the coordinating center and the block size was unknown to the clinical centers. Informed written consent was obtained prior to study participation and the study was approved by the Ethical Committee at each of the participating centers. The study has been described in detail elsewhere (see Petersen et al., 2006; Sørensen et al., 2006).

### Analysis

### Phenotypes

Prior to randomization to the weight loss intervention and after completion of the intervention participants underwent a clinical investigation protocol starting at 8.00 a.m. after a 12 h overnight fast. The first clinical investigation was preceded by a 3-day dietary run in period during which participants had to keep their habitual diet, and avoid excessive physical activity and alcohol consumption. The second clinical investigation was conducted in the 10^{th} week after start of the dietary weight loss intervention program.

Anthropometrics and body composition were assessed after the subjects voided their bladder. Body weight was measured on calibrated scales. WC was measured with the participant wearing only non-restrictive underwear. Body height was measured with a calibrated stadiometer. The mean of three measurements was recorded for each variable. Fat mass (FM) and fat-free mass (FFM) were assessed by multi frequency bio-impedance (Bodystat®; QuadScan 4000, Isle of Man, British Isles).

Resting metabolic rate was measured by ventilated hood systems routinely used at each center, and a standardized validation program was used to facilitate pooling of the results from the different centers. Energy expenditure (EE) was calculated according to the equation of Weir (see Weir, 1949). FO was calculated according to the equations of Frayn. At the second clinical investigation hood measurements were only carried out in a subset (˜⅔) of participants completing the intervention.

After participants resting supine for 15 minutes venous blood samples were drawn to determine fasting plasma glucose and fasting plasma insulin. Plasma glucose concentrations (ABX diagnostics, Montpellier, France) were measured on a COBAS MIRA automated spectrophotometric analyzer (Roche diagnostica, Basel, Switzerland). Plasma insulin concentrations were measured with a double antibody radio-immunoassay (Insulin RIA 100, Kabi-Pharmacia, Uppsala, Sweden). Homeostasis model assessment (HOMA) was used to estimate HOMA-β and HOMA-IR (see Bonora et al., 2000; Emoto et al., 1999; Matthews et al., 1985).

### Genotyping

Samples of buffy coat were sent on dry ice to the Steno Diabetes Center in Copenhagen, where DNA was extracted. Extracted DNA samples were diluted in Tris/EDTA buffer to a stock DNA solution of 100 ng/μl and a working DNA solution of 10 ng/μl. Stock solutions were stored at −80° C., working solutions were stored at 4° C. DNA samples were stored and handled in locations free of contaminating polymerase chain reaction products. High-throughput genotyping of the FTO rs9939609 SNP was performed using Taqman allelic discrimination (KBioScience, Herts, UK). DNA samples were available for 764 subjects. There was a 96.9% genotyping success rate (n=734) and the genotyping error rate was 0.27%.

### Statistical Modelling.

Examination of Hardy-Weinberg equilibrium was carried out while taking into account centre differences by summing up Pearson X² statistics for each centre and comparing with a X² distribution with 8 degrees of freedom.

First, general genetic models with no assumptions about genetic mode of transmission, i.e. no assumption of a specific effect of the risk allele (A) in individuals heterozygous and homozygous for the A allele compared with non-carriers (null hypothesis), were analyzed. The results from these analyses were used to decide whether to proceed analyzing models assuming a particular genetic mode of transmission and a particular effect of the gene variant compared with the non-carrier: dominant effect (non-carrier=0, heterozygous and homozygous=1), co-dominant effect (assessed as an additive effect: non-carrier=0, heterozygous=1, homozygous=2), or recessive effect of risk allele (non-carrier=0, heterozygous=0, homozygous=1). Firstly, models examining the main effects of rs9939609 were made followed by models examining interaction between genotype and diet.

Δweight measured in kg, ΔFM (kg), ΔFFM (kg), ΔWC measured in cm, ΔHOMA-β, ΔHOMA-IR, ΔEE measured in kcal/24 h and ΔFO % were calculated by subtracting measurement at the completion of the intervention from the measurement recorded immediately before randomization. The effects of rs9939609 genotype (TT, AT or AA) and assigned hypo-energetic diet (HF or LF) on mean Δweight, ΔFM, ΔFFM, ΔWC, ΔHOMA-β, ΔHOMA-IR, ΔEE, ΔFO % and drop out with 95%-CIs were analyzed by separate linear or logistic (drop-out) regression models. Drop-out was additionally analysed in time-to-event analysis in order to get a more detailed examination of the effect of genotype and diet on drop-out. Nelson-Aalen cumulative hazard function was used to examine if genotype and diet affected the timing of drop-out. As there was no apparent difference in drop-out timing between diets, a Cox proportional hazards model was performed with intervention time in weeks as underlying timescale in order to compare hazards for drop-out. The Breslow method was used to handle tied failures. The proportional hazards assumption was tested based on Schoenfeld residuals.

In models for changes in phenotypes it was controlled for respective baseline values (linear; separate effect of weight, WC, FM and FFM for men and women; FM, HOMA-β and HOMA-IR were log transformed), age (linear in models of ΔFFM, ΔHOMA-β, HOMA-IR, ΔEE, and ΔFO %; linear and squared in models of Δweight, ΔFM and ΔWC), sex and centre (Gaussian random effect). Difference in drop-out according to genotype and randomised diet combined was analysed adjusting for baseline BMI (linear), age (linear), sex and centre (Gaussian random effect).

Assessment of main effects was conducted by including the genotypes as covariates and as a separate covariate for the diet group to which the participants had been randomized. Gene-diet interaction analyses were carried out by estimating the genotype specific difference in mean Δweight, ΔFM, ΔFFM, ΔWC, ΔHOMA-β, ΔHOMA-IR, ΔEE and ΔFO % respectively—adjusted as described above—between LF and the HF and then comparing the differences in mean Δweight, ΔFM, ΔFFM, ΔWC, ΔHOMA-β, ΔHOMA-IR, ΔEE and ΔFO % respectively for AT and/or AA with TT or TT and AT depending on assumed genetic mode of transmission.

The statistical software STATA version 9.0 (Stata, College Station, Texas, United States) was used for all statistical analyses.

### Results

SNP rs9939609 was genotyped successfully in 734 of the (771) obese subjects randomized to one of the two diets. The rs9939609 allele frequencies were in Hardy-Weinberg equilibrium (P=0.97).

In randomising subjects to the two hypo-energetic diets SNP rs9939609 genotype was not distributed evenly. Wild-type (T/T) subjects were more likely to be randomised to the high fat diet (55.2%) than the low fat diet (44.8%), heterozygotes (NT) were distributed evenly between the two diets and homozygotes for the risk allele (NA) were more likely to be randomised to the low fat diet (59.2%) than to the high fat diet (40.8%).

In total, 115 of the successfully genotyped subjects failed to complete the 10-week weight loss intervention. Drop-out rates varied with SNP rs9939609 genotype and hypo-caloric diet and there was significant interaction between genotype and hypo-caloric diet in relation to drop-out from the intervention (see Table 1). In wild-type subjects the drop-out rate was higher for those randomised to low fat diet (21.4%) than for those randomised to the high fat diet (14.6%). For carriers of the variant A allele drop-out rates were higher on high fat diet (NT: 17.8%; NA: 28.3%) than on low fat diet (NT: 6.7%; A/A: 16.9%). Adjusting for baseline BMI, age, sex and centre significant separate effects of both FTO rs9939609 genotype (P=0.01) and hypo-energetic diet (P=0.046) on odds for drop-out were found and there was significant interaction between genotype and diet (P for interaction=0.001). The effect of combined genotype and diet group did not follow a specific genetic mode of transmission (neither dominant, nor co-dominant, nor recessive). Among subjects randomized to LF hypo-energetic diet the odds for drop-out were significantly higher for subjects with TT (OR=3.69; CI: 1.73-7.86) and AA (OR=3.32; CI: 1.40-7.82) genotypes compared to heterozygous subjects. On the HF diet subjects homozygous for the T allele (wild-type) had the lowest odds for drop-out, followed by heterozygous subjects and subjects homozygous for the A allele. For heterozygous subjects there was a significant difference in odds for drop-out between LF and HF diet; drop-out was significantly higher for subjects randomized to HF diet compared with LF diet (OR=3.08; 95%-CI: 1.50-6.31). For the TT and AA genotypes there was a trend toward lower odds for drop-out on HF compared with LF diet for TT subjects (OR=0.72; 95%-CI: 0.37-1.42) and higher odds for drop-out on HF compared with LF diet for AA subjects (OR=1.70; 95%-CI: 0.71-4.09). The cumulative hazard functions revealed no apparent difference in drop-out timing (data not shown). The results form the Cox proportional hazards model showed a pattern similar to those from the logistic regression (data not shown).

Among subjects who completed the intervention and for whom FTO rs9939609 was genotyped statistically significant interactions between genotype at FTO rs9939609 and hypo-energetic diet in relation to ΔHOMA-β (P for interaction=0.008; n=612), ΔHOMA-IR (P for interaction=0.047; n=612), and ΔEE (P for interaction=0.01; n=387) were found. The effects of FTO rs9939609 on change in phenotype were dominant.

Combined effects of FTO rs9939609 genotype and fat- and carbohydrate (CHO) content of hypo-energetic diet on decrease in insulin secretion (ΔHOMA-β) with 95%-CI for comparison with TT subjects on low-fat, high-CHO diet (LF) showed that the mean ΔHOMA-β was 9.09. Estimates were from linear regression adjusting for baseline HOMA-β (logarithm transformed; linear), age (linear), sex and centre (Gaussian random effect). Genetic mode of transmission was modelled as dominant. The mean ΔHOMA-β was measured as decrease in ΔHOMA-β relative to TT genotype on LF diet. The greatest decrease was seen in non-carriers of the A allele randomized to LF diet. Non-carriers on HF diet, carriers of the A allele on LF diet and carriers of the A allele on HF diet all experienced a significantly smaller ΔHOMA-β (19.1, p=0.006; 18.1, p=0.004; 14.5, p=0.024), but were not significantly different from each other (p=0.68) (data not shown).

Combined effects of FTO rs9939609 genotype and fat- and carbohydrate (CHO) content of hypo-energetic diet on decrease in insulin resistance (ΔHOMA-IR) with 95%-CI for comparison with TT subjects on low-fat, high-CHO diet (LF) showed a similar effect pattern as was seen for ΔHOMA-β (i.e. ΔHOMA-IR (mean=0.34)). However, the difference was only significant for non-carriers on LF diet compared with carriers of the A allele on LF diet (0.44, p=0.01). Estimates were from linear regression adjusting for baseline HOMA-IR (logarithm transformed; linear), age (linear), sex and centre (Gaussian random effect). Genetic mode of transmission was modelled as dominant.

In the group of 387 subjects for whom data on EE was available at baseline and post-intervention, mean ΔEE was 114 kcal/24 h. Again the decrease in fasting EE (kcal/24 h) was relative to TT genotype on LF diet. Combined effects of FTO rs9939609 genotype and fat- and carbohydrate (CHO) content of hypo-energetic diet on decrease in fasting energy expenditure (ΔEE) were measured with 95%-CI for comparison with TT subjects on low-fat, high-CHO diet (LF). Estimates were from linear regression adjusting for baseline fasting EE (linear), age (linear), sex and centre (Gaussian random effect). Genetic mode of transmission was modelled as dominant. The decrease was significantly greater for non-carriers randomized to the HF diet (82 kcal/24 h, p=0.005) and carriers of the A allele on either diet (LF: 75 kcal/24 h, p=0.005; HF: 68 kcal/24 h, p=0.01) compared with non-carriers on LF diet. Additional adjustment for change in BMI produced similar associations between genotype and diet combined and ΔHOMA-β, ΔHOMA-IR and ΔEE, respectively.

No effect of FTO rs9939609 on Δweight, ΔFM, ΔFFM, ΔWC or ΔFO % neither alone nor in combination with fat- and CHO content of hypo-energetic diet was found. Mean Δweight among successfully genotyped participants completing the intervention was 6.80 kg consisting of 5.35 kg ΔFM and 1.47 kg ΔFFM. WC decreased on average by 6.33 cm, while FO % increased by 2.5 percent point from 46.6% to 49.1% of EE.

In the present weight loss intervention study drop-out was affected both by FTO rs9939609 genotype and by the macronutrient content of the hypo-energetic diet (HF vs. LF) and there was interaction between genotype and diet. The results suggest that low fat/high carbohydrate hypo-energetic diet is preferred to high fat/low carbohydrate hypo-energetic diet for carriers of the FTO rs9939609 allele due to lower odds for drop-out. Wild-type subjects tend to be more likely to drop-out on a low fat/high carbohydrate hypo-energetic diet than high fat/low carbohydrate hypo-energetic diet. Among heterozygous subjects odds for drop-out was significantly higher when randomized to HF diet than when randomized to LF diet.

For subjects completing the intervention we found statistically significant interactions between FTO rs9939609 genotype and fat- and CHO content of the hypo-energetic diet in relation to change in three (ΔHOMA-β, ΔHOMA-IR, and ΔEE) of eight obesity-related phenotypes. The effects of FTO were dominant and similar for ΔHOMA-β, ΔHOMA-IR, and ΔEE. For A allele non-carriers the improvement in HOMA-β and HOMA-IR was greater and the decrease in EE was smaller on LF than HF diet. ΔHOMA-β, ΔHOMA-IR, and ΔEE in non-carriers on LF diet were also different from ΔHOMA-β, ΔHOMA-IR, and ΔEE in A allele carriers on LF diet and HF diet. For A allele carriers there was no significant difference in ΔHOMA-β, ΔHOMA-IR, and ΔEE between diets.

Given the strong association between FTO and obesity the absence of an effect of rs9939609 on Δweight, ΔWC, ΔFM, or ΔFFM could be explained in that the FTO gene is a stability gene rather than a susceptibility gene and works its influence on body fatness early in life. The effect of FTO on body composition and on the risk of obesity and overweight is observed already in childhood and persists into adolescence. While no association is observed with birth weight or with the ponderal index at birth, already at the age of two weeks FTO SNPs are associated with increased weight and ponderal index.

TABLE 1
Drop out rates according to diet* and genotype at SNP rs9939609.

| | FTO rs9939609 genotype | | |
|---|---|---|---|
| | TT | AT | AA |
| | n = 249 | n = 354 | n = 130 |
| low fat,/high CHO diet | 21.4% | 6.7% | 16.9% |
| high fat/low CHO diet | 14.6% | 17.8% | 28.3% |

*Randomised to either low fat/high CHO, hypo-energetic diet (LF) or high fat/low CHO hypo-energetic diet (HF) in a 10 wk, parallel two arm intervention study.

### REFERENCES

Bonora E, Targher G, Alberiche M, Bonadonna R C, Saggiani F, Zenere M B, Monauni T and Muggeo M (2000), Homeostasis model assessment closely mirrors the glucose clamp technique in the assessment of insulin sensitivity: studies in subjects with various degrees of glucose tolerance and insulin sensitivity. Diabetes Care 23:57-63.
Dina C, Meyre D, Gallina, S, Durand E, Koerner A, Jacobsen P, Carlsson L M S, Kiess W, Vatin V, Lecoeur C, Delplanque J, Vaillant E, Pattou F, Ruiz J, Weill J, Levy-Marchal C, Horber F, Potoczna N, Hercberg S, Le Stunff C, Bougneres P, Kovacs P, Marre M, Balkau B, Cauchi S, Chevre J-C and Froguel P (2007), Variation in FTO contributes to childhood obesity and severe adult obesity. Nat. Genet. 39:724-726.
Emoto M, Nishizawa Y, Maekawa K, Hiura Y, Kanda H, Kawagishi T, Shoji T, Okuno Y and Morii H (1999), Homeostasis model assessment as a clinical index of insulin resistance in type 2 diabetic patients treated with sulfonylureas. Diabetes Care 22:818-822.
Fraylling T M, Timpson N J, Weedon M N, Zeggini E, Freathy R M, Lindgren C M, Perry J R B, Elliott K S, Lango H, Rayner N W, Shields B, Harries L W, Barrett J C, Ellard S, Groves C J, Knight B, Patch A-M, Ness A R, Ebrahim S, Lawlor D A, Ring S M, Ben-Shlomo Y, Jarvelin M-R, Sovio U, Bennett A J, Melzer D, Ferrucci L, Loos R J F, Barroso I, Wareham N J, Karpe F, Owen K R, Cardon L R, Walker M, Hitman G A, Palmer C A N, Doney A S F, Morris A D, Smith G D, The Wellcome Trust Case Control Consortium, Hattersley A T and McCarthy M I (2007), A common variant in the FTO gene is associated with body mass index and predisposes to childhood and adult obesity. Science 316:889-894.
Gerken T, Girard C A, Tung Y-C L, Webby C J, Saudek V, Hewitson K S, Yeo G S H, McDonough M A, Cunliffe S, McNeill L A, Galvanovskis J, Rorsman P, Robins P, Prieur X, Coll A P, Ma M, Jovanovic Z, Farooqi I S, Sedgwick B, Barroso I, Lindahl T, Ponting C P, Ashcroft F M, O'Rahilly S and Schofield C J (2007), The obesity-associated FTO gene encodes a 2-oxoglutarate-dependent nucleic acid demethylase. Science 318:1469-1472.
Haupt A, Thamer C, Machann J, Kirchhoff K, Stefan N, Tschritter O, Machicao F, Schick F, Haring H-U and Fritsche A (2008), Impact of Variation in the FTO Gene on Whole Body Fat Distribution, Ectopic Fat, and Weight Los. Obesity (Silver Spring) 16:1969-1972.
Matthews D R, Hosker J P, Rudenski A S, Naylor B A, Treacher D F and Turner R C (1985), Homeostasis model assessment: insulin resistance and beta-cell function from fasting plasma glucose and insulin concentrations in man. Diabetologia 28:412-419.
Müller T D, Hinney A, Scherag A, Nguyen T T, Schreiner F, Schäfer H, Hebebrand J, Roth C L and Reinehr T (2008), Fat mass and obesity associated gene (FTO): no significant association of variant rs9939609 with weight loss in a lifestyle intervention and lipid metabolism markers in German obese children and adolescents. BMC. Med. Genet. 9:85.
Petersen M, Taylor M A, Saris W H M, Verdich C, Toubro S, Macdonald I, Rossner S, Stich V, Guy-Brand B, Langin D, Martinez J A, Pedersen O, Holst C, Sørensen T I A, Astrup A and The Nugenob Consortium (2006), Randomized, multi-center trial of two hypo-energetic diets in obese subjects: high- versus low-fat content. Int. J. Obes. (Lond.). 30:552-60.
Scuteri A, Sanna S, Chen W-M, Uda M, Albai G, Strait J, Najjar S, Nagaraja R, Orru M, Usala G, Dei M, Lai S, Maschio A, Busonero F, Mulas A, Ehret G B, Fink A A, Weder A B, Cooper R S, Galan P, Chakravarti A, Schlessinger D, Cao A, Lakatta E and Abecasis G R (2007), Genome-wide association scan shows genetic variants in the FTO gene are associated with obesity-related traits. PLoS Genetics 3:1200-1210.
Sørensen T I A, Boutin P, Tayloer M A, Larsen L H, Verdich C, Petersen L, Holst C, Echwald S M, Dina C, Tourbo S, Petersen M, Polak J, Clement K, Martinez J A, Langin D, Oppert J M, Stch V, Macdonald I, Amer P, Saris W H M, Pedersen O, Astrup A, Froguel P and The Nugenob Consortium (2006), Genetic polymorphisms and weight loss in obesity: a randomised trial of hypo-energetic high- versus low-fat diets. PLoS Clinical Trials 1(2):e12.
Weir J B (1949), New methods for calculating metabolic rate with special reference to protein metabolism. J. Physiol. 109:1-9.

1. A method for assessing whether an individual will complete a low fat/high carbohydrate diet or a high fat/low carbohydrate diet, the method comprising the step of determining the genotype of the SNP rs9939609 in the FTO gene, wherein:
(i) the presence of either one or two A allelic forms of the SNP rs9939609 is indicative of a increased likelihood that the individual completes a low fat/high carbohydrate diet compared to a high fat/low carbohydrate diet, and
(ii) the absence of an A allelic form of the SNP rs9939609 is indicative of a increased likelihood that the individual completes a high fat/low carbohydrate diet compared to a low fat/high carbohydrate diet.

2. A method according to claim 1, wherein the individual is overweight or obese.

3. A method according to claim 1, wherein the high fat/low carbohydrate or low fat/high carbohydrate diet is a hypo-energetic diet.

4. A method for making a dietary weight intervention program for an individual, the method comprising the steps of:
(i) identifying if the individual has at least one or no A allelic forms of the SNP rs9939609 in the FTO gene,
(ii) using a low fat/high carbohydrate diet in the making of a dietary weight loss intervention program suitable for treating an individual which has been identified as having at least one A allelic form of the SNP rs9939609 in the FTO gene and using a high fat/low carbohydrate diet in the making of a dietary weight intervention program suitable for treating an individual which has been identified as having no A allelic form of the SNP rs9939609 in the FTO gene.

5. A method according to claim 4, wherein the individual is overweight or obese.

6. The use of SNP rs9939609 in the FTO gene for selecting an optimal diet for an individual.

7. The use according to claim 6, wherein the individual is overweight or obese.
